(19) United States
(12) Patent Application Publication
(10) Pub. No.: US 2016/0206781 A1
(43) Pub. Date: Jul. 21, 2016

(54) METHODS AND COMPOSITIONS FOR THE PRODUCTION OF COMPOSITES FOR BONE IMPLANTATION

(71) Applicants: Zurab KAKABADZE, Tbilisi (GE); David CHAKHUNASHVILI, Tbilisi (GE); Konstantine CHAKHUNASHVILI, Tbilisi (GE); Nicholas KIPSHIDZE, Tbilisi (GE); Ann KAKABADZE, Tbilisi (GE)

(72) Inventors: Zurab KAKABADZE, Tbilisi (GE); David CHAKHUNASHVILI, Tbilisi (GE); Konstantine CHAKHUNASHVILI, Tbilisi (GE); Nicholas KIPSHIDZE, Tbilisi (GE); Ann KAKABADZE, Tbilisi (GE)

(21) Appl. No.: 14/983,283

(22) Filed: Dec. 29, 2015

Related U.S. Application Data
(60) Provisional application No. 62/097,148, filed on Dec. 29, 2014.

(51) Int. Cl. A61L 27/36; A61L 27/50; B65B 63/08; A61L 27/38

(52) U.S. Cl. CPC A61L 27/3608; A61L 27/3834; A61L 27/3821; A61L 27/3691; A61L 27/3687; A61L 27/50; B65B 63/08; A61L 2430/02; A61L 2400/18

(57) ABSTRACT

The invention disclosed herein relates to methods and compositions useful for the production of composites for bone implantation. The invention includes methods for production of lyophilized bone fragments seeded with lyophilized stem cells, mesenchymal stem cells, bone marrow stem cells, periosteal cells or osteocytes that are derived either from the recipient of the bone implant or from allogeneic sources. The methods of the invention comprise production of bone implant material including the steps of cutting solid bone into fragments, decellularizing the bone fragments, seeding stem cells, mesenchymal stem cells, bone marrow stem cells, periosteal cells or osteocytes onto the decellularized bone fragments and lyophilizing the complex of bone fragments and cells. The stabilized bone matrix and complex of bone and cells increases the ease of transport, storage and reconstitution of bone and cells for bone implantation.

### CROSS REFERENCE TO RELATED APPLICATIONS

This application claims the benefit of U.S. Provisional Application No. 62/097,148, filed Dec. 29, 2014, which is hereby incorporated in its entirety by reference.

### BACKGROUND OF THE INVENTION

1. Field of the invention

The invention relates to methods and compositions useful for the production of composites for bone implantation. The incorporation of stem cells or cells that are capable of forming bone into bone implant material have been shown to improve bone implantation success, as measured by increased volume of mineralized matrix, increased bone mineral density, increased bone volume fraction, increased osteoid deposition, increased proximity of bone proteins to vascular networks, increased vascularization of bone, increased bone strength, increased cells expressing bone specific proteins and markers and reduced inflammatory response and immune cell infiltration (Lee et al., 2010 and Sava-Rosianu et al., 2013).

An embodiment of the invention comprises methods for producing composites of lyophilized and decellularized bone fragments seeded with lyophilized stem cells, bone marrow stem cells, mesenchymal stem cells, periosteal cells or osteocytes. The invention provides for methods and compositions for production of a stabilized bone matrix seeded with lyophilized stem cells, bone marrow stem cells, mesenchymal stem cells, periosteal cells or osteocytes that are derived either from the recipient of the bone implant or from allogeneic sources. The stabilized bone matrix and complex of bone and cells increases the ease of transport, storage and reconstitution of bone and cells for bone implantation.

The invention provides for methods and compositions where the bone implant material is seeded with cells derived either from the recipient of the implant or from allogeneic sources. In addition, the invention provides for methods and compositions for production of a lyophilized bone matrix seeded with lyophilized stem cells, mesenchymal stem cells, bone marrow stem cells, periosteal cells or osteocytes than can be used for forming a specific geometric shape in the recipient of the bone implant. The invention also provides compositions for bone grafts that can be used with other biocompatible matrices or scaffolds.

2. Description of the Related Art

Mesenchymal stem cells (MSC) improve bone graft and bone implantation success when incorporated into bone implants, biocompatible matrices or scaffolds (Correia et al., 2011). MSCs promote vascular development and osteoinductive processes to increase osteocyte presence or osteoid deposition in bone implants or bone grafts leading to improved bone strength, bone mineralization and vascularization. The present invention provides improved methods and compositions for the production of bone implant material comprising stem cells, mesenchymal stem cells, bone marrow stem cells, periosteal cells or osteocytes that is highly stable and adaptable.

### SUMMARY OF THE INVENTION

Disclosed herein are methods and compositions for the production of bone implant material. The invention includes methods for production of lyophilized bone fragments seeded with stem cells, mesenchymal stem cells, bone marrow stem cells, periosteal cells or osteocytes. The methods of the invention include production of bone implant material including the steps of cutting solid bone into fragments, decellularizing the bone fragments, seeding stem cells, mesenchymal stem cells, bone marrow stem cells, periosteal cells or osteocytes onto the decellularized bone fragments and lyophilizing the complex of bone fragments and cells. In one aspect a method of the invention includes lyophilizing the bone fragments prior to seeding the cells on the bone fragments. In another aspect, the invention includes lyophilizing the bone fragments after seeding the cells onto the bone fragments. In yet another aspect, the invention includes lyophilizing the bone fragments before and after seeding the cells. In one aspect, the stem cells, mesenchymal stem cells, bone marrow stem cells, periosteal cells or osteocytes are human. In another aspect, the cells are collected from the recipient of the bone implant. In yet another aspect, the cells are collected from an allogeneic source. In an embodiment of the invention, the decellularized and lyophilized bone with lyophilized stem cells, bone marrow stem cells, periosteal cells or osteocytes is reconstituted and used with another biocompatible matrix or scaffold. In another embodiment, the decellularized and lyophilized bone with lyophilized stem cells, bone marrow stem cells, periosteal cells or osteocytes is shaped specifically to fit the recipient of the implant. In another aspect of the invention, the decellularized and lyophilized bone with lyophilized stem cells, bone marrow stem cells, periosteal cells or osteocytes is shaped using a machine and/or digitized clinical images.

In a variation of the invention, the bone fragments are derived from cattle. In another variation of the invention, the bone fragments are treated with heparin. In a variation of the invention, the bone fragments are decellularized using organic solvents, such as acetone, a mixture of chloroform and ethanol or a mixture of chloroform and methanol. In another variation, the bone fragments are treated with a detergent, such as Triton X-100 or Sodium dodecyl sulfate. In another variation, the bone fragments are demineralized prior to decellularization. In a variation, the demineralization step comprises use of an acid, such as hydrochloric acid. In yet another variation of the invention, the bone fragments are purified to remove blood components. In an embodiment, the purification step includes use of a solution of hydrogen peroxide. In another embodiment, the bone fragments are deproteinized. In yet another embodiment, the bone fragments are deproteinized using sodium hypochlorite. In an aspect of the invention the bone fragments are heated at 120° C. to 200° C. for less than 5 hours. In another aspect, the lyophilized bone fragments and stem cells, bone marrow stem cells, periosteal cells or osteocytes are sterilized in a gamma chamber or an ultraviolet light chamber. In yet another aspect, the lyophilized bone fragments and stem cells, bone marrow stem cells, periosteal cells or osteocytes are packaged by blister packaging.

In an aspect of the invention the stem cells, bone marrow stem cells, periosteal cells or osteocytes are lyophilized in culture medium, such as Dulbecco's modified Eagle's Medium or Phosphate Buffered Saline. In another aspect of the invention, the stem cells, bone marrow stem cells, periosteal cells or osteocytes are lyophilized in osteoinductive medium wherein the medium contains bone morphogenetic protein-7, beta-glycerophosphate, dexamethasone, insulin growth factor, platelet-derived growth factor or transforming growth factor beta. In yet another aspect of the invention, the lyophilized stem cells bone marrow stem cells, periosteal cells or osteocytes are reconstituted in osteoinductive medium containing bone morphogenetic protein-7, beta-glycerophosphate, dexamethasone, insulin growth factor, platelet-derived growth factor or transforming growth factor beta.

An embodiment of the invention are compositions for bone implantation that include decellularized and lyophilized bone fragments with lyophilized bone marrow stem cells, bone marrow stem cells, periosteal cells or osteocytes.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an example of a flow chart describing the steps performed in an embodiment of the invention to produce a lyophilized complex of lyophilized bone fragments and mesenchymal stem cells.

FIG. 2A is an image of a representative bone fragment produced by cutting solid bone.

FIG. 2B is an image of a decellularized bone fragment.

FIG. 2C is an image of a lyophilized and decellularized bone fragment.

FIG. 2D is a Computer tomography (CT) image of a decellularized bone fragment.

FIG. 3A is an image of a lyophilizer.

FIG. 3B is an image of a lyophilized and decellularized bone fragment.

FIG. 3C is an image of a cell culture incubator with mesenchymal stem cells.

FIG. 3D is an image of a lyophilized and decellularized bone fragment being seeded with mesenchymal stem cells growing with culture medium in a pitri dish.

FIG. 4A is an image of frozen mesenchymal stem cells and culture medium in a petri dish with Dulbecco's modified Eagle's Medium.

FIG. 4B is an image of lyophilized mesenchymal stem cells and lyophilized Dulbecco's modified Eagle's Medium.

FIG. 4C is an image of lyophilized mesenchymal stem cells that have been rehydrated/reconstituted.

FIG. 4D is a scanning electron micrograph image of lyophilized mesenchymal stem cells before rehydration/reconstitution.

FIG. 4E is a scanning electron micrograph image of lyophilized mesenchymal stem cells after rehydration/reconstitution.

FIG. 4F is a micrograph image of lyophilized mesenchymal stem cells that have been rehydrated a bone specific marker protein, CD 105.

FIG. 5A is a micrograph image of lyophilized, decellularized bovine bone with lyophilized rat bone marrow stem cells that have been rehydrated and stained for a bone specific marker protein, Bone Morphogenetic protein-2 (BMP-2).

FIG. 5B is a micrograph image of lyophilized, decellularized bovine bone with lyophilized rat bone marrow stem cells that have been rehydrated and transplanted and stained for a bone specific marker protein, Bone Morphogenetic protein-2 (BMP-2) after transplantation.

FIG. 6A is an image of the lyophilized/freeze-dried complex of mesenchymal stem cells (MSC) seeded onto decellularized and lyophilized bone.

FIG. 6B is an image of reconstituted/rehydrated complex of lyophilized MSC and decellularized and lyophilized bone prior to transplantation.

FIG. 6C is a scanning electron micrograph image of lyophilized MSC and decellularized and lyophilized bone prior to rehydration.

FIG. 6D is a scanning electron micrograph image of reconstituted/rehydrated complex of lyophilized MSC and decellularized and lyophilized bone prior to transplantation.

FIG. 7A is an image of a bone fragment after bone collection and carving.

FIG. 7B is an image of a bone fragment undergoing decellularization in an organic solvent.

FIG. 7C is an image of a bone fragment after decellularization.

FIG. 7D is an image of bone fragments that have been manually shaped to the form of a mandible branch.

FIG. 8 is a diagram of the experimental design of the studies performed using the mandibular defect model in rats.

FIG. 9A is an image of the defect introduced to the mandible.

FIG. 9B is an image of rehydrated, lyophilized bone with rehydrated, lyophilized MSC (Freeze-dried complex) attached to titanium plates.

FIG. 9C is an image of rehydrated, lyophilized bone with rehydrated, lyophilized mesenchymal stem cells (Freeze-dried complex) attached to titanium plates and inserted into the mandible defect introduced to the rats.

FIG. 9D is an X-ray image of a mandibular transplant, 1 month post-transplantation.

FIG. 10A is an X-ray image of a mandibular transplant, 3 months post-transplantation.

FIG. 10B is an image produced using contrast angiography of a mandibular transplant, 3 months post-transplantation.

FIG. 11 is a graph demonstrating the expression of bone specific genes 10 days after transplantation with rehydrated, lyophilized bone and mesenchymal stem cells.

FIG. 12A is a micrograph of transplanted bone tissue, 5 days after transplantation of rehydrated lyophilized complex of MSC and decellularized bone, exhibiting inflammation and newly formed blood vessels.

FIG. 12B is a micrograph of transplanted bone tissue, 1 month after transplantation of rehydrated lyophilized complex of MSC and decellularized bone, exhibiting forming bone with osteoclasts and osteoblasts around the newly forming bone.

FIG. 12C is a micrograph of transplanted bone tissue, 3 months after transplantation of rehydrated lyophilized complex of MSC and decellularized bone, exhibiting increased osteogenesis.

FIG. 12D is a micrograph of transplanted bone tissue, 6 months after transplantation of rehydrated lyophilized complex of MSC and decellularized bone, exhibiting complete bone formation.

### DETAILED DESCRIPTION OF THE INVENTION

The methods of the invention comprise a protocol for the preparation of a composite comprising lyophilized bone fragments and lyophilized stem cells, bone marrow stem cells, mesenchymal stem cells, periosteal cells or osteocytes. An embodiment of the invention comprises the steps of fragmentation of the bone, decellularization of the bone fragments, purification purification of the bone fragments, lyophilization of the bone fragments, seeding of the bone fragments with cells, lyophilization of the bone fragment and cell complex and sterilization and packaging of the bone fragment and cell complex. Below is an exemplary protocol for an embodiment of the invention.

Cutting and Processing of Bone

Cattle bone is cut with a special saw into fragments. The bone fragments may be 10×2×2 cm in size. The cattle bone fragment size can vary according to the size needed for the recipient of the bone graft and/or the shape of the bone implant needed. These bone fragments are placed in deionized water solution containing heparin for 24 hours to remove blood components, which are presented in the bone. Afterwards, the bone fragments are rinsed with 200 ml 0.9% saline solution and frozen at −80° C. for at least 12 hours (fragments are fully placed in the solution). During the night, the frozen fragments of the bone are thawed at 4° C. and rinsed with PBS. After, the fragments are placed in the stirrer and rinsed with distilled H₂O containing SDS (Sigma) for 72 hours: rinsing starts with 0.01% SDS solution for 24 hours, which is followed by rinsing with 0.1% SDS solution for another 24 hours and rinsing with 1% SDS solution for final 24 hours. Then, bone fragments are rinsed with distilled H₂O for 15 minutes and followed with 1% Triton X-100 (Sigma) for 30 minutes to remove the remaining SDS. Decellularized bone fragments are then rinsed with PBS for 4 hours.

Bone fragments are placed in the stirrer and rinsed with the solution containing chloroform and ethanol, to remove the remaining fatty matters. The solution of chloroform and ethanol is used with following ratio: chloroform to ethanol ratio 2:1 for the first 24 hours and 1:2 for the second 24 hours.

To remove the solvent which is left in defatted bone fragments, deionized water with a 50:1 ratio is added, and afterwards the solution is stirred at 120 rpm for 12 hours, which will remove the remaining solvent from the fragments. Deionized water is changed every 2 hours with fresh deionized water, which increases the rinsing efficiency. The rinsed bone fragments are dried at 37° C. for 24 hours.

Alternatively, decellularized bone fragments can be prepared by rinsing cut bone fragments in Tris-NaCl solution for 6 hours. Afterwards, the bone fragments may be demineralized. The demineralization process of the bone fragments may be performed using 0.6 M HCl for 15 minutes. The demineralized bone fragments are then decellularized in either acetone for 17 hours or chloroform/methanol solution for 6 hours, then rinsed in distilled water for 12 hours at room temperature. The bone fragments are then chemically sterilized in absolute ethanol for 24 hours, then transferred into ethanol 80%, 70%, 20% solution within 24 h for each step. The residual ethanol is eliminated by washing with sterile PBS for 24 h.

Deproteinization of Bone

On the next stage, the bone fragments are placed in the stirrer (to remove the protein that is found in the bone and for inactivation of prions, which causes cattle spongiform encephalopathy) and at 120 rpm the bone fragments are rinsed with 4% sodium hypochlorite for 24 hours. To remove the remaining solvents from the deproteinized bone, deionized water is added and stirred at 120 rpm for 72 hours, which removes residual sodium hypochlorite. The deionized water, for the 1st 12 hours is changed every 2 hours and afterwards deionized water is changed every 12 hours. Afterwards, bone fragments are processed with 5% hydrogen peroxide for 6 hours, which will remove non-collagen protein molecules and, at the same time, will deteriorate such substances as pigments, remaining lipids, toughly dissolving salts etc. After this, bone fragments are rinsed in deionized water for 10 hours and the deionized water is changed every 2 hours.

Thermal Processing of Decellularized Bone Fragments

Defatted and deproteinized fragments are thermally processed at a high temperature. The temperature in the heat chamber is increased by 2° C. every minute and a temperature of 600° C. is maintained for 3 hours. The chamber is then cooled. After this thermal processing stage the bone fragments are ready to serve as a matrix for seeding cells.

Analysis of Decellularized Bone Fragments

DNA analysis, histochemical and microbiological studies may be conducted on all decellularized bone fragments. Density and porosity may be analyzed.

Bone Marrow Stem Cell Isolation and Seeding

Stem cells obtained from human bone marrow (hBMSCs) populations is extracted by the processing of the femoral head of patients according to the following method. Under the sterile conditions of the operating room, the femoral heads are segmented transversally into two hemispheres to expose the trabecular bone. Cells are then extracted from the trabecular bone with successive washes with phosphate buffered saline solution (PBS) (Gibco, USA) to facilitate the disaggregation of the tissue. The trabecular bone is then mechanically dissected to obtain fragments of approximately 2 mm³. The obtained solution from each hemisphere is recollected and filtered with a 70 μm cell strainer (Falcon, USA) before centrifuging at 400 g for 10 min. Cell pellets are resuspended in non-osteogenic medium consisting of Dulbecco's modified Eagle's Medium (DMEM) (Sigma, USA), supplemented with 10% Fetal Bovine Serum (FBS) (Gibco, USA) and 1% Antibiotics (streptomycin and penicillin) (Gibco, USA), and cultured in 25 cm² flasks at 37° C. in a humidified atmosphere containing 5% CO₂. Afterwards, the cultures are washed with PBS to remove the non-adherent cells and further expanded until ˜80% confluence, and then are harvested and expanded in 75 cm² flasks. After subculture, these cells are designated to be seeded on the decellularized and deproteinized bone fragments.

Periosteal Cell Isolation and Seeding

Harvesting mandibular periosteal tissues must be held under general anesthesia, a full-thickness mandibular periosteal biopsy. At the site, a full-thickness flap must be generated by using a blunt periosteal elevator without damaging the “osteogenic” inner layer of the periosteum. An intact periosteal sheet measuring 5×5 mm, must be separated from the underlying bone. The harvested tissues must be immediately transferred to the laboratory under sterile conditions. After rinsing the periosteum thoroughly with PBS containing 100 U/mL penicillin and 100 μg/mL streptomycin, the biopsies must be minced in small pieces and digested in 0.5% type II collagenase (Worthington Biochemical Corporation, Lake Wood, N.J.) for 4 hours at 37° C. The isolated cells must be centrifuged, resuspended in complete media supplemented with FGF/Dex, plated in a 56 cm² dish, and cultured in a humidified 37° C./5% CO₂ incubator. Afterwards, these cells must be designated to be seeded on the decellularized and deproteinized bone fragments.

Lyophilization Stage

After processing the bone, deptoteinization, conducting thermal processes, preparing the bone marrow stem cells or periosteal cells and seeding on the bone matrix, the composite of the above mentioned, must be freeze-dried using a lyophilizer. The water is removed from the composite of lyophilized bone and lyophilized cells by sublimation of frozen ice, i.e. converting it to steam, passing the liquid phase. After the lyophilization, the lyophilized composite of bone and cells is wrapped in a blister packaging and, afterwards, sterilized in gamma chamber with dosage of 25 kGy. Alternatively, the composite is sterilized using an ultraviolet light chamber.

Advantages and Utility

Briefly, and as described in more detail below, described herein are compositions and methods for improving the success of bone implantation. The invention is useful for creating bone grafts and implants to reconstruct bone from conditions comprising, congenital defects, cancer resections, periodontal disease and trauma.

Several features of the current approach should be noted. The methods and compositions of the invention promote osteogenesis, osteoconductivity, osteoinductivity and osseointegration of bone implants. The incorporation of stem cells or cells that are capable of forming bone into bone implant material improves bone implantation success, as measured by increased volume of mineralized matrix, increased bone mineral density, increased bone volume fraction, increased osteoid deposition, increased proximity of bone proteins to vascular networks, increased vascularization of bone, increased bone strength, increased cells expressing bone specific proteins and markers and reduced inflammatory response and immune cell infiltration. The present invention provides improved methods and compositions for the production of bone implant material comprising stem cells, mesenchymal stem cells, bone marrow stem cells, periosteal cells or osteocytes that is highly stable and adaptable.

The bone implant material can be made in various solid shapes and forms depending on individual demand to fix bone defects caused by different reasons (such as: oncologic, physical and other causes). The various shapes can be created specifically to meet the needs of the recipient of the bone implant. The invention includes methods and compositions for creating specific geometric shapes of the implant material using a machine and digitized clinical images. The cells that are seeded in the bone fragments can be derived from the recipient of the bone implant, improving the success of the bone implantation and reduced inflammatory responses after implantation. The compositions of the invention can be used with hydroxyapatite or other bone implantation biocompatible materials and matrices such as: collagen, fibrin, fibrinogen, thrombin, chitosan, alginate, tricalcium phosphate, macroporous biphasic calcium phosphate, poly(lactic-co-glycolic acid), porous poly(epsilon-caprolactone-c-l-lactide sponges) and pullalan/dextran polysaccharide.

An important advantage is the stability and ease of storage and transportation of the lyophilized complex of lyophilized, bone fragments seeded with cells. Reconstitution of the lyophilized complex of bone fragments and cells can be performed rapidly and the percentage of surviving cells from the lyophilization after reconstitution is high. The surviving cells seeded in the bone fragments improve the implantation success as measured by bone strength, bone mineralization, ability of bone implant to express specific bone proteins/markers, improved cellular infiltration and vascularization and reduced inflammatory response.

Definitions

Terms used in the claims and specification are defined as set forth below unless otherwise specified.

The term “bone implant” refers to implanted material that promotes bone regeneration alone or in combination with other substances in the recipient of the implanted material through osteogenesis, osteoinduction, osteopromotion and osteoconduction, in combination or alone.

The term “bone implantation” or “bone grafting” refers to the surgical procedure that replaces missing or damaged bone with a bone implant.

The term “bone marrow stem cells” refers to multipotent stem cells derived from the bone marrow, including mesenchymal stem cells and hematopoietic stem cells.

The term “mesenchymal stem cells” refers to multipotent stem cells derived from the bone marrow stroma and have the ability to differentiate into osteoblasts.

The term “periosteal cells” refers to cell derived from the periosteum or outer service of bones and can include cells derived from the cambium layer of the periosteum, including progenitor cells that develop into osteoblasts.

The terms “bone matrix composite”, “composite of bone matrix” or “complex of lyophilized bone fragments and mesenchymal stem cells” refers to the mixture of lyophilized bone fragments seeded with lyophilized mesenchymal stem cells and may be used to refer to rehydrated or reconstituted lyophilized bone matrix composite or dehydrated or unreconstituted lyophilized bone matrix composite.

The term “decellularization” refers to removal or lysis of cells from a substance.

The term “seeding” refers to adding cells to or onto a substance or mixing cells with a substance.

The terms “lyophilizing” and “freeze-drying” refer to a dehydration process typically used to preserve a perishable material by freezing the material and then reducing the surrounding pressure to all the frozen water in the material to sublimate directly from the solid phase to the gas phase.

The term “reconstitution” refers to adding a sufficient amount of a solution, such as, but not limited to, culture medium or a buffered salt solution, to allow for rehydration of lyophilized substances, such as adding sufficient volume of liquid culture medium or phosphate buffered saline to lyophilized cells and lyophilized bone to adequately rehydrate the cells and tissue to allow for the cells to be viable upon transplantation or culture in vitro.

The term “osteoinductive” refers to stimulation of osteoprogenitor cells to differentiate into osteocytes, including osteoblasts.

The term “bone fragment” refers to cut up pieces of solid bone of any size, volume or shape and can include small bone granules and can be derived from any organism.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

Abbreviations used in this application include the following: “MSC” refers to mesenchymal stem cells, “DMEM” refers to Dulbecco's Modified Eagle's Medium.

It must be noted that, as used in the specification and the appended claims, the singular forms “a,” “an” and “the” include plural referents unless the context clearly dictates otherwise.

Methods of the Invention

Methods for creating bone grafts and implants to reconstruct bone from conditions comprising, congenital defects, cancer resections, periodontal disease and trauma are also encompassed by the present invention. Said methods of the invention include methods for cutting, deproteinization, purification, decellularization and lyophilization of bone fragments. Methods of the invention also comprise shaping composites of lyophilized bone and cells for repair of a bone defect.

Compositions of the Invention

The compositions of the invention can be prepared for bone implantation and bone grafting surgical procedures. These compositions can comprise, reconstituted complex of decellularized and lyophilized bone fragments with lyophilized stem cells, bone marrow stem cells, periosteal cells or osteocytein. The lyophilized complex of bone fragments and cells can be reconstituted with a saline solution, such as phosphate buffered saline or any other solution at buffered at physiological pH. The reconstituted complex can contain osteoinductive compounds such as bone morphogenetic protein (such as bone morphogenetic protein-7), beta-glycerophosphate, dexamethasone, insulin growth factor, platelet-derived growth factor and transforming growth factor beta or other materials well known to those skilled in the art. Such materials should be non-toxic and should not interfere with the efficacy of the reconstituted bone fragments and cells. The precise nature of the carrier or other material can depend on the site of bone implantation. The composition can be composed of bone from cattle, human or another organism. The composition can comprise cells from human or another organism. The cells can be derived from the recipient of the bone implant or from an allogeneic source. The complex of bone fragments and cells can be shaped into a specific geometry best suited for the specific needs of the recipient of the bone implant. The complex can be shaped prior or after reconstitution of the lyophilized complex of bone fragments and cells.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds, Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

METHODS:

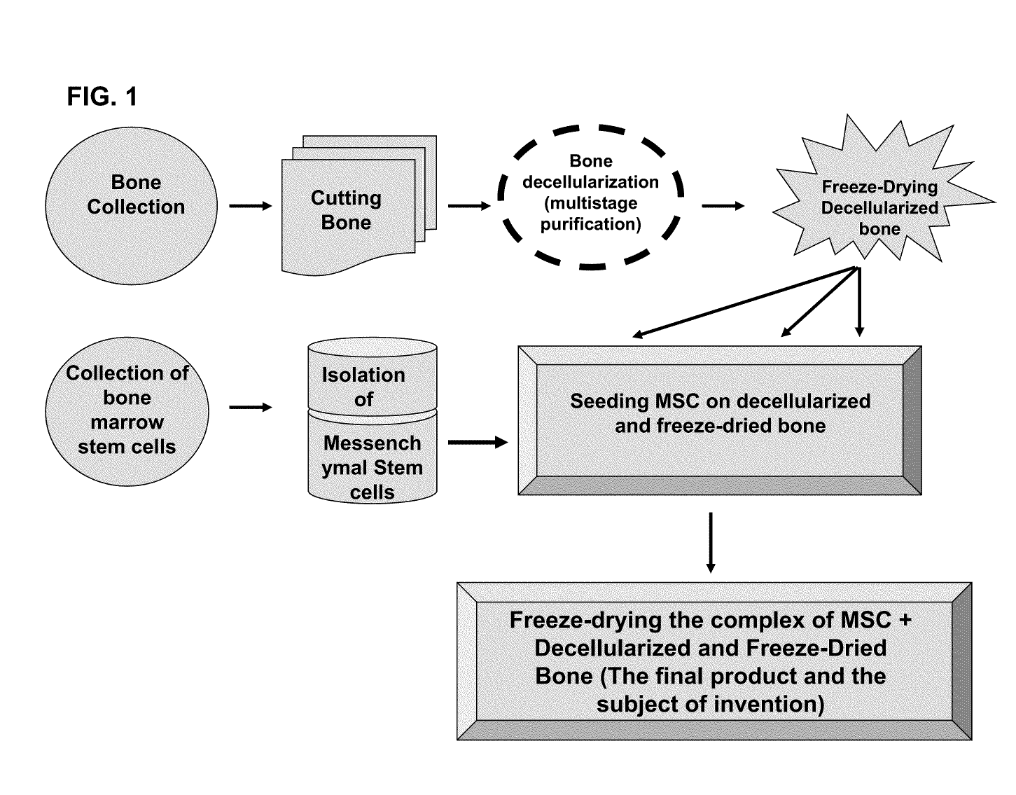

Exemplary methods of the invention include methods for cutting, deproteinization, purification, decellularization and lyophilization of bone fragments and are described in more detail below. FIG. 1 depicts an exemplary embodiment of the invention diagraming the steps of preparing a complex of reconstituted lyophilized and decellularized bone fragments seeded with mesenchymal stem cells for bone repair procedures. Briefly, decellularized and lyophilized bone fragments are placed in Dulbecco's Modified Eagles Medium for the seeding of mesenchymal stem cells. Mesenchymal stem cells are added to the decellularized and lyophilized bone fragments and cultured. After 4 days of culture, the complex of lyophilized bone fragments and mesenchymal stem cells are frozen at −45 to −60° C. and lyophilized. The lyophilized complex of decellularized bone and mesenchymal stem cells are packaged. When needed, the lyophilized complex can then be rehydrated/reconstituted and prepared for transplantation.

Immunohistochemistry was performed on sections of lyophilized bone and mesenchymal stem cells before and after rehydration. Antibody IHC Staining: The slides were deparaffinized and rehydrated to water. Antigen retrieval were performed using steam and proteinase K digestion methods. After antigen retrieval, the slides were allowed to cool at room temperature for 20 minutes prior to the next step. Then the slides were washed in three changes of PBS for 5 minutes each and the blocked with 3% H₂O₂. After washing in three changes of PBS, the slides were incubated in primary antibody (CD105/Endoglin at 1:100, BMP-2 at 1:100, Collagen Ial at 1:100 and Fibronectin at 1:200) diluted with IHC-Tek Antibody Diluent for 1 hour at room temperature. The slides were then washed three times in PBS and incubated with biotinylated secondary antibody for 30 minutes. The slides were washed in PBS and then incubated with HRP-Streptavidin for 30 minutes. Then incubate with DAB chromogen substrate solution for 5-10 minutes and then wash with PBS and counterstained with Mayer's hematoxylin. Green color is Anti-BMP2 Antibody (aa86-102) IHC-plus™ LS-B785. Red is the secondary antibody.

For Quantitative PCR (Q-PCR) of bone tissue cells, total RNA from the bone tissue was purified using miRNeasy mini kit according to the manufacture's instruction (Qiagen). cDNA was synthesized using the iScript cDNA synthesis Kit (BioRad). Q-PCR was carried out with iTaq universal SYBR green supermix (BioRad) on a 7500 Fast Real-Time PCR system (Life Technologies). 18S rRNA was used as internal control for gene expression normalization.

A rat mandibular defect model was used to examine the effect of transplantation of reconstituted complex of lyophilized bone and mesenchymal stem cells in vivo. Two types of bone grafts were evaluated: 1. Decellularized and Freeze-dried bone and 2. Decellularized and Freeze-Dried bone with seeded mesenchymal stem cells (Freeze-Dried Complex) for repairing critical size mandible defects on the rats. Experiments were conducted on 45 Lewis Rats. Animals were divided in equivalent groups (15 in each group). In all groups animals underwent a mandible defect creation procedure.

### EXAMPLES

### Example 1

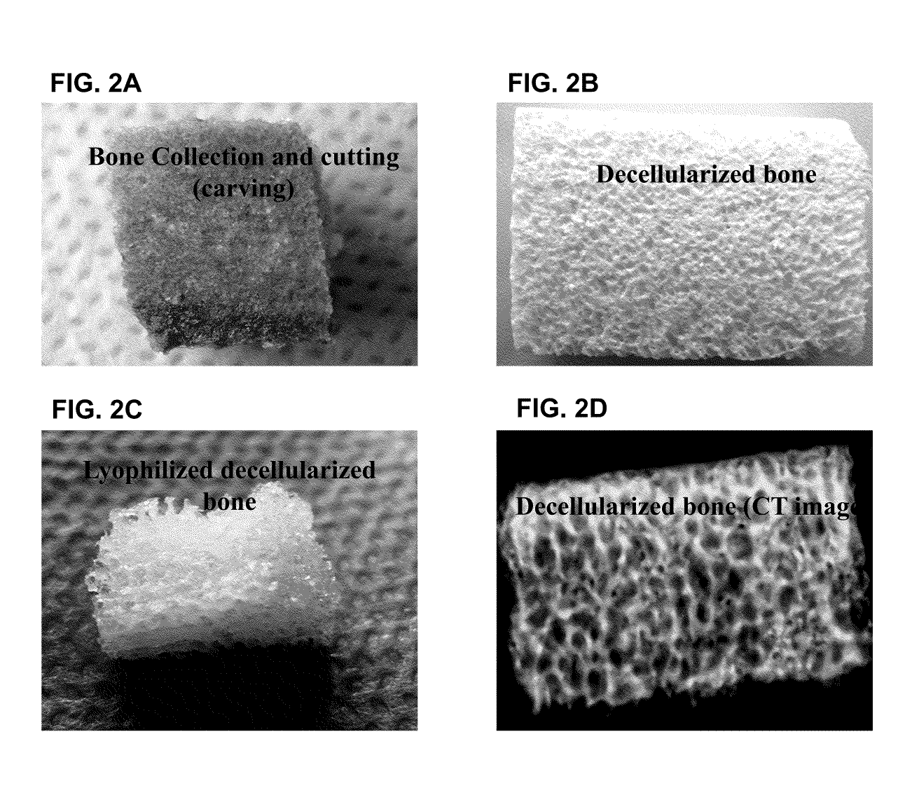

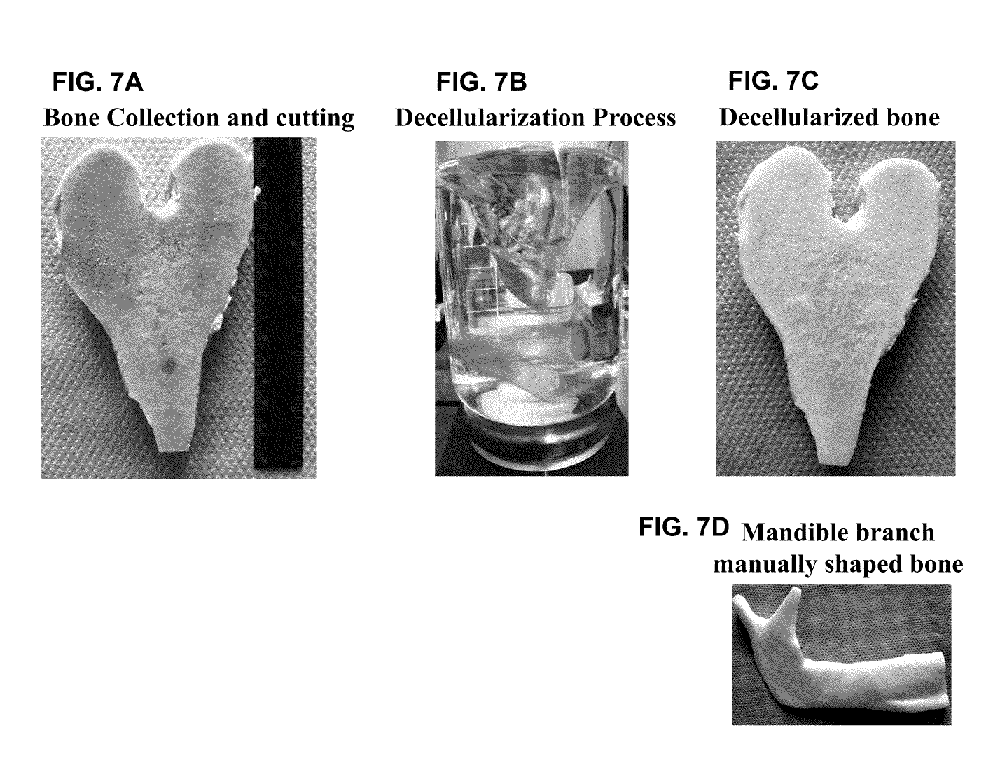

Decellularization and deproteinization of bone fragments. Bovine femur is cut into desired fragments (FIG. 2A and 7A). The bone fragments are then processed to decellularize and deproteinize the fragments (FIG. 7B). Bone fragments are placed into a solution containing deionized water and heparin for 24 hours. Bone fragments are then rinsed with 200 ml 0.9% saline solution. The bone fragments are then frozen at −80° C. for a minimum of 12 hours while fully covered in a 0.9% saline solution. The bone fragments are then thawed overnight at 4° C. Afterwards, the bone fragments are rinsed with PBS. Next, the bone fragments are washed 1-3 times with distilled H₂O containing 0.01% sodium dodecyl sulfate for 24-48 hours, while stirring. Afterwards, bone fragments are rinsed with distilled H₂O for 15 minutes followed by rinsing with a solution of 1% Triton X-100 (Sigma) for 30 minutes. Bone fragments are then rinsed with PBS for 4 hours. The bone fragments are next rinsed twice with a chloroform and ethanol solution (chloroform/ethanol=2:1) while in the stirrer for 24 hours. Afterwards, deionized water is added to the chloroform/ethanol solution to generate a water/chloroform and ethanol solution=50/1. The bone fragments are then rinsed with deionized water 5-7 times for 2 hours at 120 rpm. The decellularized bone fragments are then dried at 37° C. for 24 hours to produce dried decellularized bone (FIG. 2B and FIG. 2D).

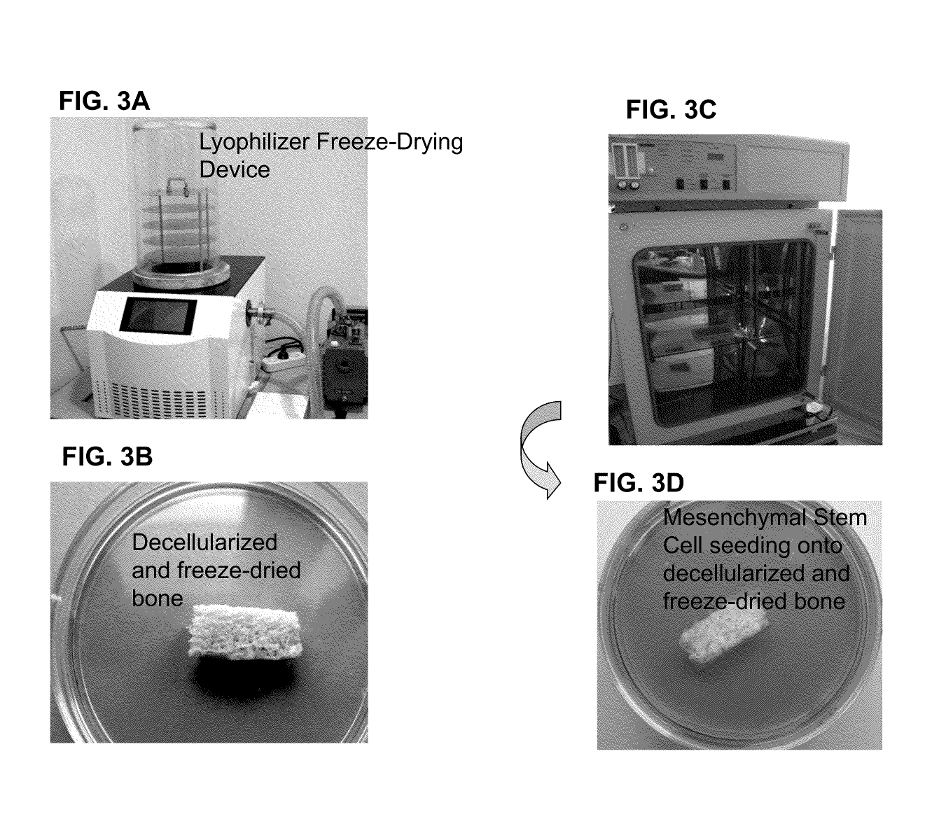

The bone fragments are then treated for deproteinization. The bone fragments are rinsed with 4% sodium hypochlorite solution for 24 hours, followed by rinsing 8-12 times with deionized water for 2 hours at 120 rpm for each rinse and changing water with fresh deionized water. Afterwards, the bone fragments are processed with 5% hydrogen peroxide for 6 hours, followed by rinsing 5 times with deionized water for 2 hours at 120 rpm. Next, the bone fragments undergo thermal processing. The bone fragments are placed in a heated chamber with temperature increasing by 2° C. every minute. The bone fragments undergo thermal processing under 120-200° C. for 3 hours. Next, the chamber is cooled to room temperature, while the bone fragments are still inside the chamber. The bone fragments are then analyzed by histochemical, microbiological and density/porosity analysis. After analysis, the bone fragments are lyophilized (FIGS. 2C, 3A and 3B).

### Example 2

Collection bone marrow stem cells and seeding of cells onto decellularized and lyophilized bone.

Bone marrow stem cells are collected and filtered with a 70 μm cell strainer. Bone marrow stem cells are then centrifuged at 400 g for 10 minutes. Cell pellets are then resuspended in non-osteogenic media containing Dublecco's modified Eagle's Medium (DMEM) (Sigma, USA), which is also supplemented with 10% Fetal Bovine Serum (FBS) (GIBCO, USA) and 1% antibiotics (Streptomycin and penicillin) (Gibco, USA). Bone marrow stem cells are then placed in a 25 cm² cell culture dish and culturing at 37° C. in a humidified atmosphere containing 5% CO₂. The cell culture is then rinsed in PBS and transferred to 75 Cm² flasks with cell culture medium (DMEM). The cell culture is then seeded into the decellularized and lyophilized bone fragment (FIGS. 3C and 3D).

### Example 3

Collection of periosteal cells and seeding of cells onto decellularized bone and lyophilized bone.

The periosteum is rinsed with PBS containing 100 U/mL penicillin and 100 μg/mL streptomycin. The periosteum is then cut into smaller pieces. Afterwards, the periosteum digested in 0.5% type II collagenase for 4 hours at 37° C. The isolated periosteal cells are then centrifuged at 400 g for 5 minutes. The isolated periosteal cells are next resuspended in FGF/Dex and placed in 56 cm² cell culture dish. The cells are then cultured in a humidified 37° C./5% CO₂ incubator for 72 hours. Afterwards, the culture is seeded onto the decellularized bone fragment(s).

### Example 4

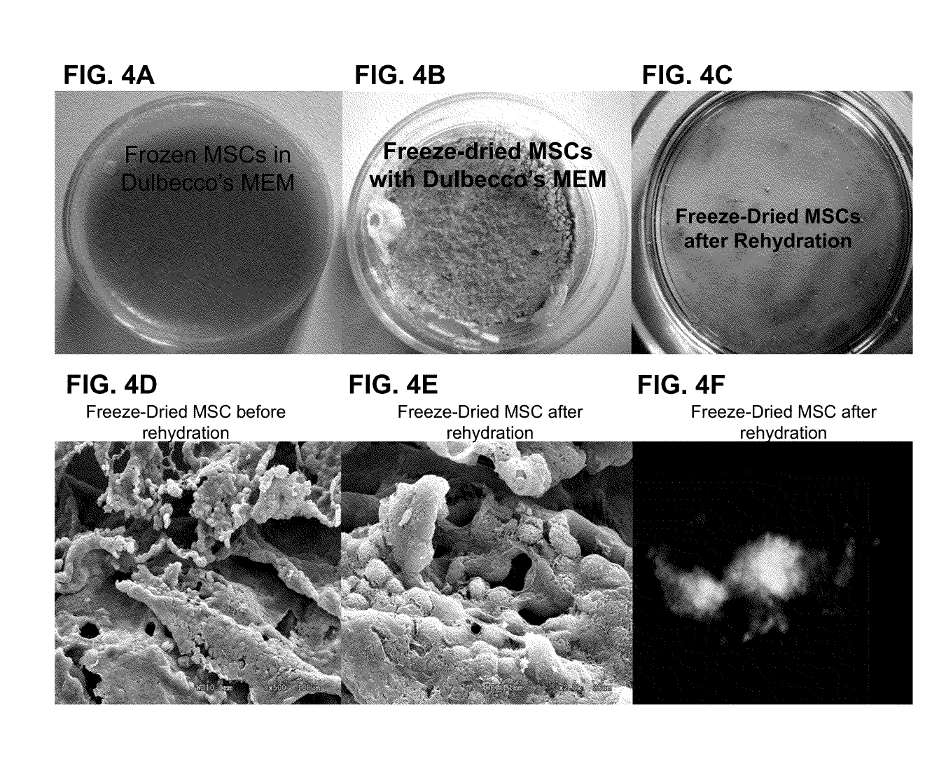

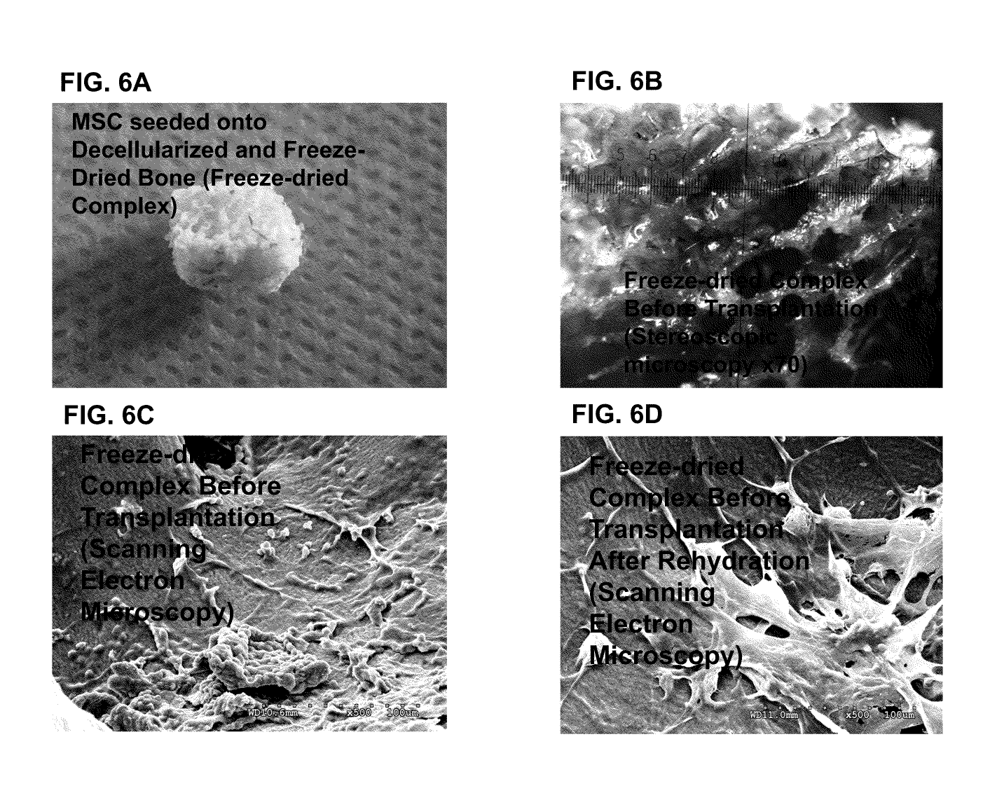

Lyophilization and sterilization of complex of bone fragments seeded with mesenchymal stem cells. The complex of decellularized bone and periosteal cells or bone marrow stem cells are placed in a lyophilizer and freeze-dried (FIGS. 4A, 4B, 4D, 6C and 6A). The temperature of the lyophilizer is set at −30 to −40° C., and the vacuum is controlled under 10-15 P (FIG. 3A). The drying procedure lasts for 9 hours. In other embodiments, the drying procedure lasts for 18-24 hours. Afterwards, the chamber is warmed up to 15 to 20° C. at a rate of 0.2° C./min and held for 6-8 h. The freeze-dried composite is then packed in blisters. Afterwards, the product is placed in a gamma chamber and sterilized in the gamma chamber with 25 kGy.

### Example 5

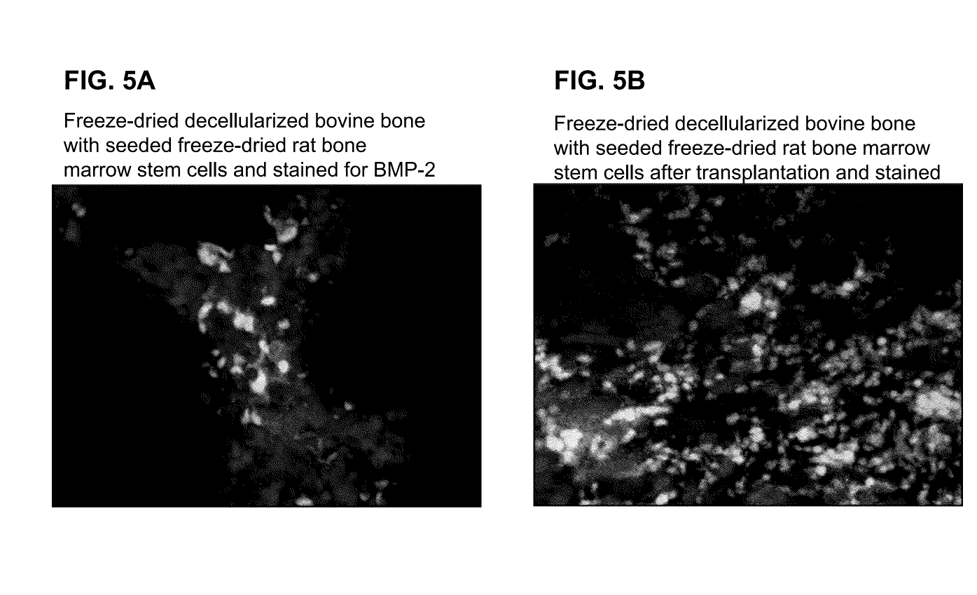

Reconstitution of lyophilized complex of bone fragments and mesenchymal stem cells. The lyophilized complex of bone fragments and mesenchymal stem cells is reconstituted or rehydrated with PBS (FIGS. 4C, 4E, 6B and 6D). The reconstituted complex expresses bone-specific markers, such as CD 105 (FIG. 4F) and bone morphogenic protein 2 (BMP-2) (FIGS. 5A and 5B).

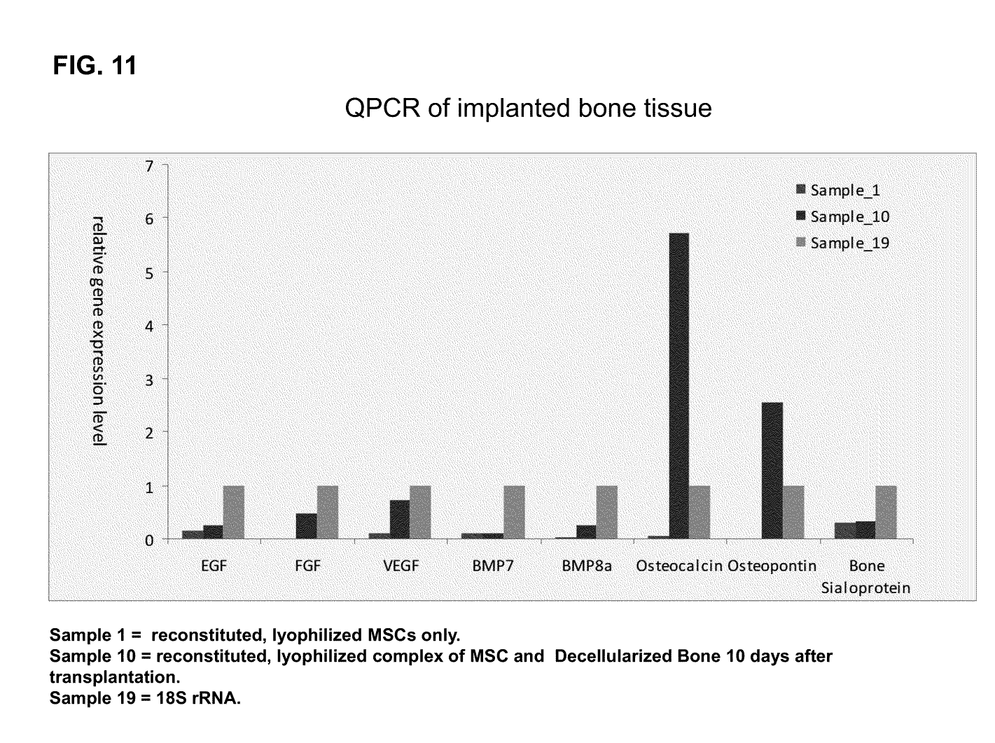

Implanted reconstituted, lyophilized bone with seeded lyophilized MSC's express bone specific maker proteins after implantation. Quantitative PCR (Q-PCR) of bone tissue was performed (FIG. 11). After 10 days of transplantation of reconstituted, lyophilized complex of bone fragments and mesenchymal stem cells, increased expression of bone-specific genes (bone morphogenetic proteins) and growth factors involved in osteogenesis was observed.

### Example 6

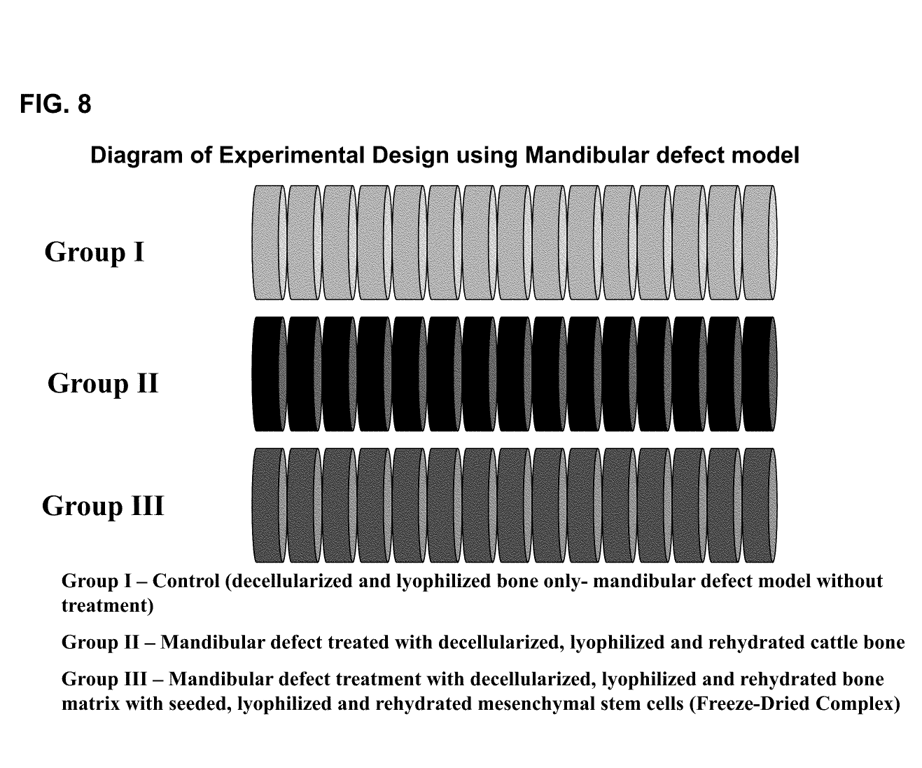

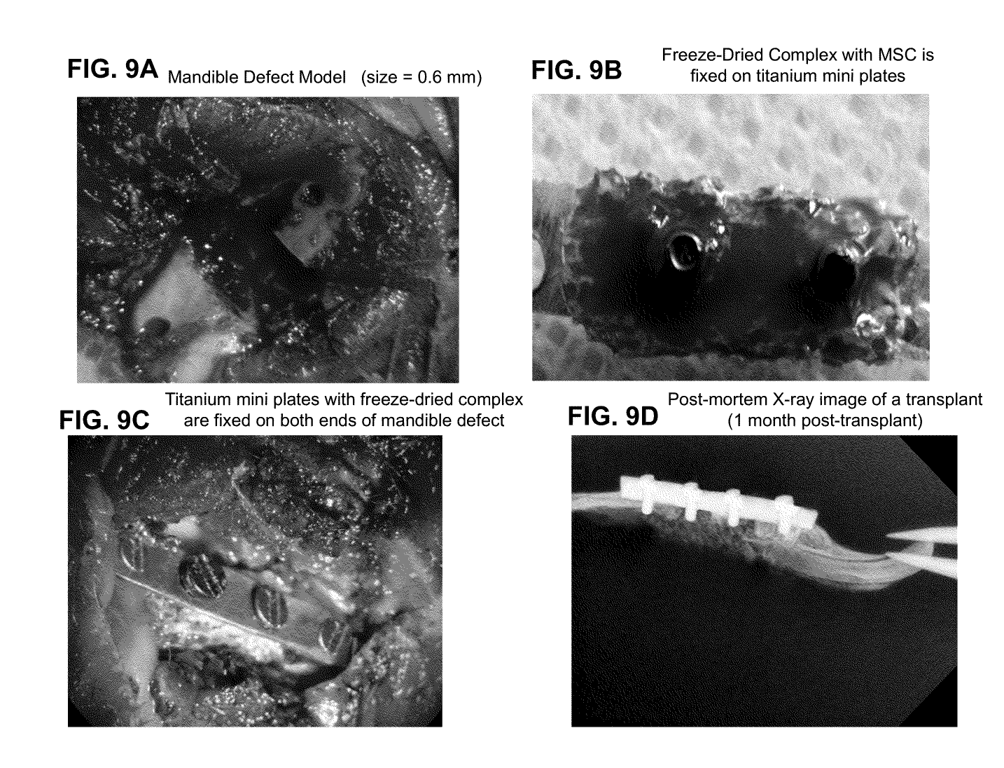

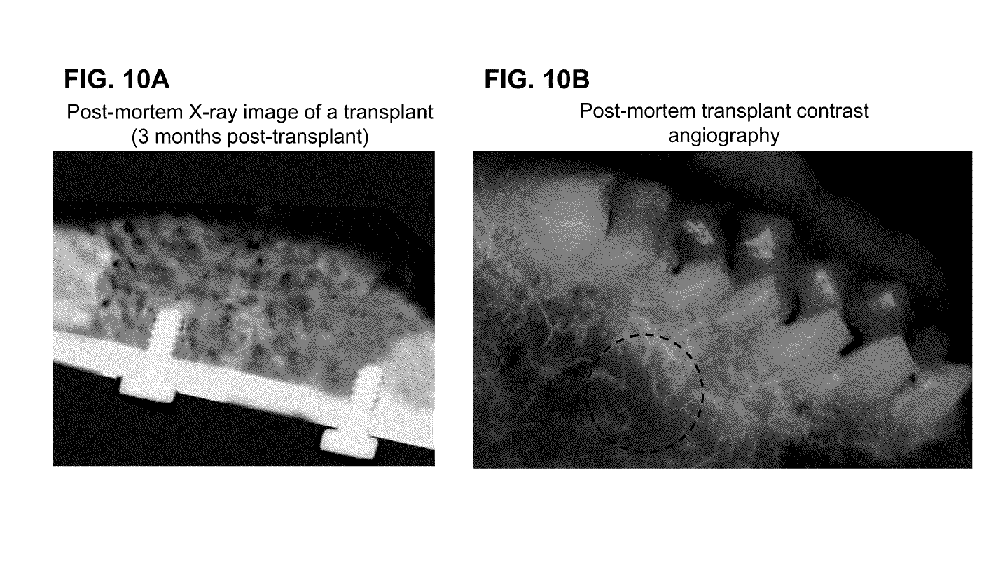

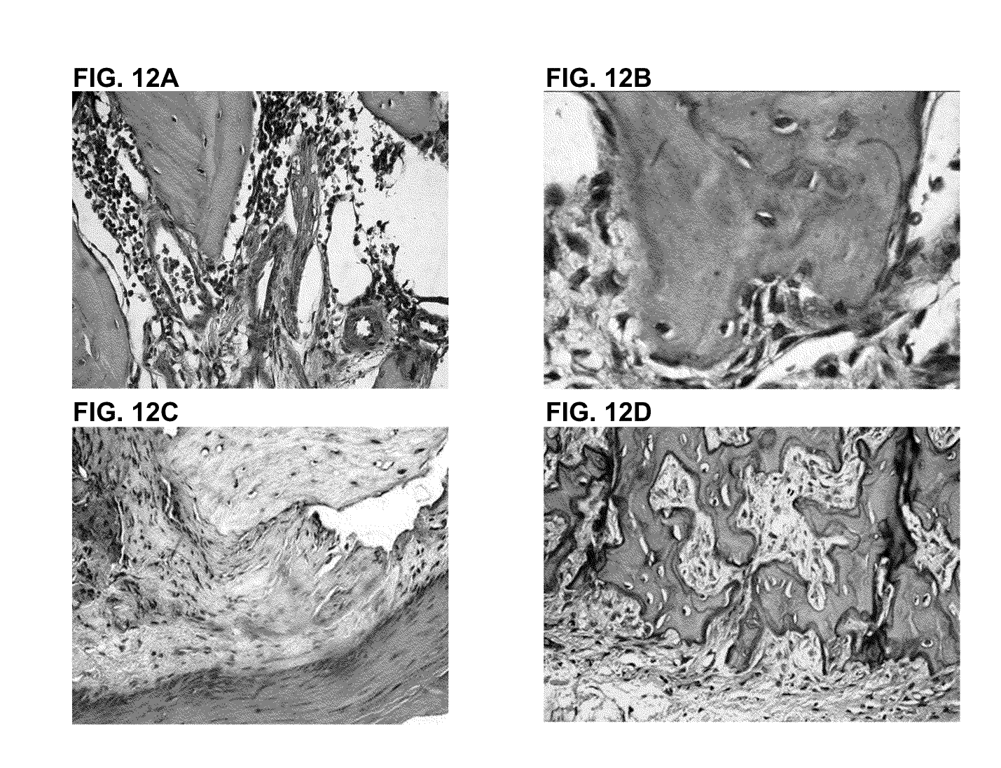

Transplantation of reconstituted lyophilized bone fragments and lyophilized mesenchymal stem cells. We evaluated and compared results from treatment with two types of bone grafts: 1. Decellularized and Freeze-dried bone and 2. Decellularized and Freeze-Dried bone with seeded mesenchymal stem cells (Freeze-Dried Complex) was used for repairing critical size mandible defects on the rats (FIGS. 8 and 9A). Experiments were conducted on 45 Lewis Rats. Animals were divided into three equal groups (15 in each group). In all groups, animals underwent a mandible defect creation procedure (FIG. 9A). The Freeze-Dried Complex was attached onto mini titanium plates to be fixed on both ends of the mandible defect (FIGS. 9B and 9C). Five days after transplant, inflammation is observed (FIG. 12A). One month after transplantation, new bone growth is observed (FIGS. 9D and 12B). Increased bone repair and growth is observed 3 months post-transplant (FIG. 10A and 12C). After 3 months post-transplant, new blood vessels are also observed (FIG. 10B). By six months post-transplant, complete bone growth and repair of the mandibular defect is observed (FIG. 12D).

### REFERENCES CITED

1. Lee S et al. Bone Regeneration Using Mesenchymal Stem Cells Loaded onto Allogeneic Cancellous Bone Granules. Tissue Engineering and Regenerative Medicine, Vol. 7, No. 4, pp 401-409 (2010).

2. Correia, C. et al. In Vitro Model of Vascularaized Bone: Synergizing Vascular Development and Osteogenesis. PLOS one, Dec. 02, 2011.

3. Sava-Rosianu R. et al. Alveolar Bone Repair Using Mesenchymal Stem Cells Placed On Granular Scaffolds in a Rat Model. Digest Journal of Nanomaterials and Biostructures, Vol. 8, No. 1, January-March 2013, p. 303-311.

1. A method of creating a bone implant for repairing bone defects, comprising:
i) cutting bone into solid fragments of bone;
ii) decellularizing bone fragments producing decellularized bone fragments;
iii) seeding stem cells, bone marrow stem cells, periosteal cells or osteocytes onto the decellularized bone fragments; and
iv) lyophilizing the bone fragments and stem cells, bone marrow stem cells, periosteal cells or osteocytes.

2. The method of claim 1, wherein the stem cells, bone marrow stem cells, periosteal cells or osteocytes are collected from a recipient of the bone implant.

3. The method of claim 1, wherein the stem cells, bone marrow stem cells, periosteal cells or osteocytes are of human allogeneic origin.

4. (canceled)

5. (canceled)

6. The method of claim 1, wherein the size of the solid fragments of bone are specifically determined for a recipient of the bone implant.

7. (canceled)

8. (canceled)

9. (canceled)

10. (canceled)

11. The method of claim 1, wherein the decellularized bone fragments are lyophilized before and after seeding stem cells, bone marrow stem cells, periosteal cells or osteocytes.

12. (canceled)

13. (canceled)

14. (canceled)

15. The method of claim 1, wherein the bone fragments are demineralized using an acid prior to decellularization.

16. (canceled)

17. The method of claim 1, wherein the decellularization step comprises use of an organic solvents.

18. (canceled)

19. (canceled)

20. The method of claim 17, wherein the organic solvent comprises a mixture of chloroform and ethanol.

21. (canceled)

22. (canceled)

23. The method of claim 17, wherein the organic solvent comprises acetone.

24. The method of claim 1, wherein the decellularization step comprises use of a detergent.

25. The method of claim 23, wherein the detergent comprises Sodium dodecyl sulfate and Triton X-100.

26. (canceled)

27. (canceled)

28. (canceled)

29. (canceled)

30. The method of claim 1, further comprising a deproteinization step.

31. The method of claim 30, wherein the deproteinization step comprises use of sodium hypochlorite.

32. The method of claim 1, further comprising a thermal processing step comprising, heating decellularized bone fragments in a heat chamber at a range of 120° C. to 200° C. for less than 5 hours.

33. (canceled)

34. The method of claim 1, further comprising a step of sterilization of the lyophilized bone fragments and stem cells, bone marrow stem cells, periosteal cells or osteocytes.

35. The method of claim 34, wherein the sterilization is performed in a gamma chamber or in an ultraviolet light chamber.

36. The method of claim 1, further comprising a step of blister packaging the bone fragments and bone marrow stem cells or periosteal cells.

37. The method of claim 1, wherein in the decellularized and lyophilized bone is from cattle.

38. The method of claim 1, wherein the solid fragments of bone are contacted with heparin.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method of repairing bone defects, comprising:
applying to a subject in need of bone repair, a composition comprising:
i) reconstituted decellularized and lyophilized bone; and
ii) reconstituted lyophilized stem cells, bone marrow stem cells, periosteal cells or osteocytes.
